(19) United States
(12) Patent Application Publication
(10) Pub. No.: US 2015/0133657 A1
(43) Pub. Date: May 14, 2015

(54) PROCESS FOR THE PREPARATION OF RIVAROXABAN

(71) Applicant: RANBAXY LABORATORIES LIMITED, New Delhi, Delhi (IN)

(72) Inventors: Pankaj Kumar Singh, Kanpur Nagar (IN); Mukesh Kumar Sharma, Gurgaon (IN); Chandra Has Khanduri, Gurgaon (IN)

(21) Appl. No.: 14/400,696

(22) PCT Filed: May 23, 2013
(86) PCT No.: PCT/IB2013/054280

§ 371 Date: Nov. 12, 2014

(30) Foreign Application Priority Data
May 24, 2012 (IN) 1591/DEL/2012

(51) Int. Cl. C07D 413/14; C07D 413/12

(52) U.S. Cl. CPC C07D 413/14; C07D 413/12
USPC 544/137; 544/146

(57) ABSTRACT

The present invention provides processes for the preparation of rivaroxaban. The present invention also provides an intermediate for the preparation of rivaroxaban.

### FIELD OF THE INVENTION

The present invention provides processes for the preparation of rivaroxaban. The present invention also provides an intermediate for the preparation of rivaroxaban.

### BACKGROUND OF THE INVENTION

Rivaroxaban chemically is 5-chloro-N-({(5S)-2-oxo-3-[4-(3-oxo-4-morpholinyl)phenyl]-1,3-oxazolidin-5-yl}methyl)-2-thiophenecarboxamide of Formula I.

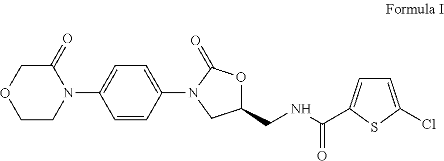 [SMILES: O=C(NC[C@H]1CN(c2ccc(N3CCOCC3=O)cc2)C(=O)O1)c1ccc(Cl)s1]

Rivaroxaban is used as an anti-thrombotic agent.

U.S. Pat. No. 7,157,456 provides rivaroxaban and processes for its preparation.

U.S. Pat. No. 8,106,192 provides a process for the preparation of N-((S)-3-bromo-2-hydroxypropyl)-5-chlorothiophene-2-carboxamide, wherein (2S)-3-aminopropane-1,2-diol hydrochloride is reacted with 5-chlorothiophene-2-carbonyl chloride to provide N-((S)-2,3-dihydroxypropyl)-5-chlorothiophene-2-carboxamide. The resulting compound is treated with hydrobromic acid in acetic acid at 21° C. to 26° C. Acetic anhydride is added and the mixture is stirred at 60° C. to 65° C. for 3 hours. Methanol is added at 20° C. to 26° C. and the reaction is stirred under reflux for 2 to 2.5 hours, then overnight at 20° C. to 26° C. to yield N-((S)-3-bromo-2-hydroxypropyl)-5-chlorothiophene-2-carboxamide, which is further converted into rivaroxaban.

U.S. Publication No. 2010/0273789 provides a process for the preparation of 5-chloro-N-[(2S)-oxiran-2-ylmethyl]thiophene-2-carboxamide, wherein ((S)-3-bromo-2-hydroxypropyl)-5-chlorothiophene-2-carboxamide (50 g, 0.167 mol) is stirred with potassium carbonate (155 g, 1.12 mol) in the presence of anhydrous tetrahydrofuran (500 mL) for three days at room temperature to give 5-chloro-N-[(2S)-oxiran-2-ylmethyl]thiophene-2-carboxamide.

U.S. Publication No. 2007/0066615 provides a process for the preparation of 5-chloro-N-((2R)-2-hydroxy-3-{[4-(3-oxo-4-morpholinyl)-phenyl]amino}propyl)-2-thiophenecarboxamide, wherein a solution of 4-(4-aminophenyl)morpholin-3-one (500 mg, 2.6 mmol) and 5-chloro-N-[(2S)-oxiranylmethyl]-2-thiophenecarboxamide (679.47 mg, 3.1 mmol) in tetrahydrofuran is stirred overnight at 60° C. in the presence of ytterbium(III) trifluoromethanesulfonate to give a precipitate, which is filtered off to provide the product in 54% yield. The remaining filtrate is concentrated and the residue obtained is purified by preparative HPLC to provide a further 38% of the product.

The prior art processes for the preparation of rivaroxaban and/or its intermediates involve long reaction times, make use of corrosive hydrobromic acid, and use expensive starting materials, catalysts, and chromatography. These processes generate corrosive hydrobromic acid as a by-product and provide the end products in low yield. Accordingly, these processes are not suitable on an industrial scale. Therefore, there is still a need in the art to develop economically attractive processes for the preparation of rivaroxaban involving the use of less expensive chemicals and having fewer reaction steps in the reaction sequence. The present inventors have developed simple, efficient, and industrially feasible processes for the preparation of rivaroxaban.

### SUMMARY OF THE INVENTION

The present invention provides processes for the preparation of rivaroxaban. The present invention also provides an intermediate for the preparation of rivaroxaban.

### DETAILED DESCRIPTION OF THE INVENTION

A first aspect of the present invention provides a process for the preparation of a compound of Formula II,

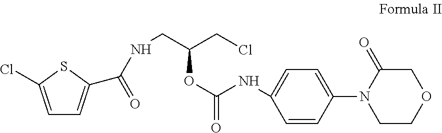 [SMILES: O=C(Nc1ccc(N2CCOCC2=O)cc1)O[C@H](CCl)CNC(=O)c1ccc(Cl)s1]

wherein the process comprises treating a compound of Formula III

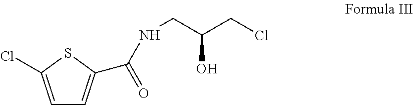 [SMILES: O=C(NC[C@H](O)CCl)c1ccc(Cl)s1]

with a compound of Formula IV

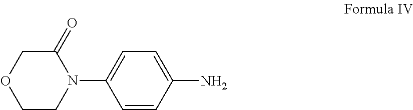 [SMILES: Nc1ccc(N2CCOCC2=O)cc1]

in the presence of phosgene or a phosgene equivalent.

A second aspect of the present invention provides a process for the preparation of a compound of Formula II,

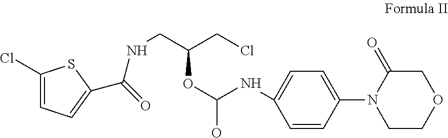 [SMILES: O=C(NC[C@@H](CCl)OC(O)Nc1ccc(N2CCOCC2=O)cc1)c1ccc(Cl)s1]

wherein the process comprises treating a compound of Formula III

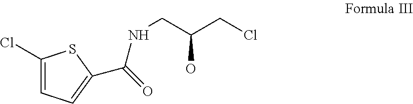 [SMILES: O=C(NC[C@H](O)CCl)c1ccc(Cl)s1]

with a compound of Formula IV

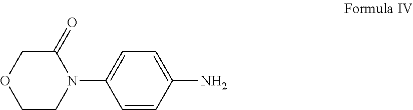 [SMILES: Nc1ccc(N2CCOCC2=O)cc1]

in the presence of phosgene or diphosgene or triphosgene.

A third aspect of the present invention provides a process for the preparation of rivaroxaban of Formula I,

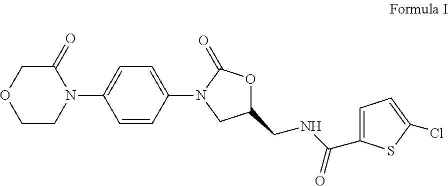

wherein the process comprises cyclization of a compound of Formula II

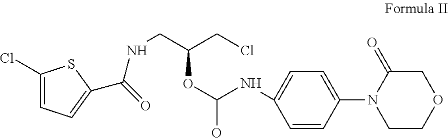 [SMILES: O=C(NC[C@@H](CCl)OC(O)Nc1ccc(N2CCOCC2=O)cc1)c1ccc(Cl)s1]

to obtain rivaroxaban of Formula I.

A fourth aspect of the present invention provides a process for the preparation of rivaroxaban of Formula I,

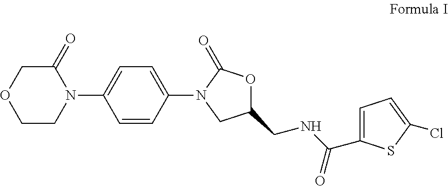

wherein the process comprises:
a) treating a compound of Formula III

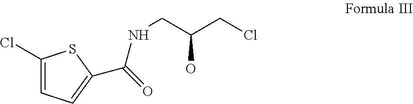 [SMILES: O=C(NC[C@H](O)CCl)c1ccc(Cl)s1]

with a compound of Formula IV

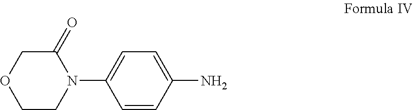 [SMILES: Nc1ccc(N2CCOCC2=O)cc1]

in the presence of phosgene or a phosgene equivalent to obtain a compound of Formula II; and

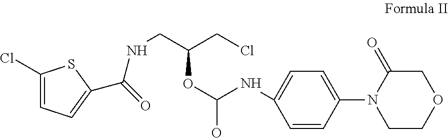 [SMILES: O=C(NC[C@@H](CCl)OC(O)Nc1ccc(N2CCOCC2=O)cc1)c1ccc(Cl)s1]

b) cyclization of the compound of Formula II

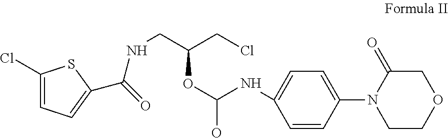 [SMILES: O=C(NC[C@@H](CCl)OC(O)Nc1ccc(N2CCOCC2=O)cc1)c1ccc(Cl)s1]

to obtain rivaroxaban of Formula I.

A fifth aspect of the present invention provides a process for the preparation of rivaroxaban of Formula I,

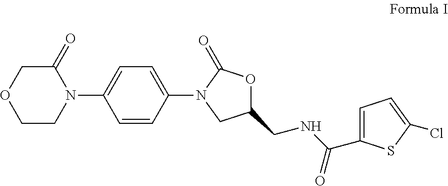

wherein the process comprises:
a) treating a compound of Formula III

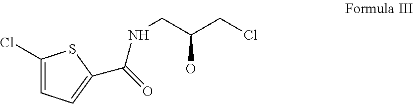 [SMILES: O=C(NC[C@H](O)CCl)c1ccc(Cl)s1]

with a compound of Formula IV

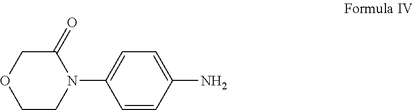 [SMILES: Nc1ccc(N2CCOCC2=O)cc1]

in the presence of phosgene or diphosgene or triphosgene to obtain a compound of Formula II; and

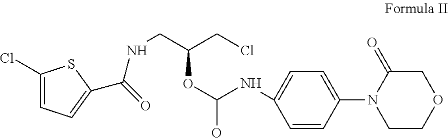 [SMILES: O=C(NC[C@@H](CCl)OC(O)Nc1ccc(N2CCOCC2=O)cc1)c1ccc(Cl)s1]

b) cyclization of the compound of Formula II

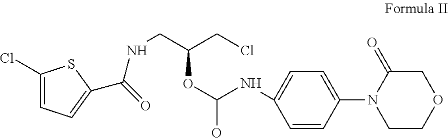 [SMILES: O=C(NC[C@@H](CCl)OC(O)Nc1ccc(N2CCOCC2=O)cc1)c1ccc(Cl)s1]

to obtain rivaroxaban of Formula I.

A sixth aspect of the present invention provides a compound of Formula II.

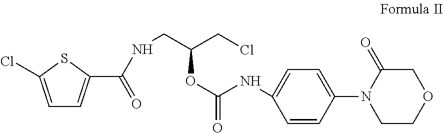 [SMILES: O=C(Nc1ccc(N2CCOCC2=O)cc1)O[C@H](CCl)CNC(=O)c1ccc(Cl)s1]

A seventh aspect of the present invention provides use of a compound of Formula II

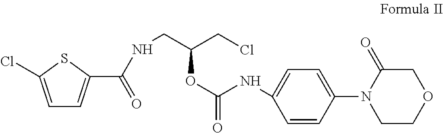 [SMILES: O=C(Nc1ccc(N2CCOCC2=O)cc1)O[C@H](CCl)CNC(=O)c1ccc(Cl)s1]

for the preparation of rivaroxaban.

The compound of Formula III may be prepared as described herein. (2S)-1-Amino-3-chloropropan-2-ol or a salt thereof, used as starting material for the preparation of the compound of Formula III, may be prepared as described herein or according to the processes provided in the art, for example, the method described in U.S. Pat. No. 6,107,519. The compound of Formula III is treated with the compound of Formula IV in a solvent in the presence of phosgene or a phosgene equivalent and optionally a base. A solution of phosgene or a phosgene equivalent in a solvent is added slowly to a mixture containing the compound of Formula III and optionally a base in a solvent prior to the treatment of the compound of Formula III with the compound of Formula IV. The phosgene equivalent may be a phosgene replacement, for example, diphosgene or triphosgene, or a carbon monoxide equivalent, for example, carbonyldiimidazole or disuccinimidyl carbonate. The solvent may be, for example, dichloromethane, dichloroethane, or a mixture thereof. The base may be, for example, pyridine, dimethylaminopyridine, triethylamine, sodium carbonate, potassium carbonate, or a mixture thereof. The mixture is stirred for about 0.5 hours to about 4 hours at about 5° C. to about 25° C. The reaction mass obtained is treated with the compound of Formula IV at about 5° C. to about 25° C. in the optional presence of a base. The base may be, for example, pyridine, dimethylaminopyridine, triethylamine, sodium carbonate, potassium carbonate, or a mixture thereof. The reaction mass is stirred for about 0.5 hours to about 6 hours at about 10° C. to about 35° C. The compound of Formula II may be isolated from the mixture by methods including layer separation, concentration, distillation, decantation, filtration, evaporation, centrifugation, or a combination thereof, and may further be dried.

The compound of Formula II is cyclized in a solvent optionally in the presence of a base at about 10° C. to about 40° C. The solvent may be, for example, acetone, acetonitrile, methanol, ethanol, isopropanol, dioxane, tetrahydofuran, water, or a mixture thereof. The base may be, for example, potassium carbonate, potassium bicarbonate, potassium hydroxide, sodium hydroxide, sodium carbonate, sodium bicarbonate, sodium hydride, or a mixture thereof. The base may be added to the mixture containing the compound of Formula II and the solvent or a mixture containing the compound of Formula II in which it is formed. The mixture is stirred for about 2 hours to about 15 hours at about 10° C. to about 40° C. The compound of Formula I may be isolated from the reaction mixture by methods including layer separation, concentration, distillation, decantation, filtration, evaporation, centrifugation, or a combination thereof, and may further be dried.

The term “about”, as used herein, when used along with values assigned to certain measurements and parameters means a variation of up to 10% from such values, or in case of a range of values, means up to a 10% variation from both the lower and upper limits of such ranges.

The term “ambient temperature”, as used herein, refers to a temperature in the range of 0° C. to 35° C.

While the present invention has been described in terms of its specific embodiments, certain modifications and equivalents will be apparent to those skilled in the art and are intended to be included within the scope of the present invention.

### EXAMPLES

### Example 1

### Preparation of (2S)-1-amino-3-chloropropan-2-ol hydrochloride

A solution of benzaldehyde (50 g, 0.540 moles) in ethanol (100 mL) was cooled to 15° C., and aqueous ammonia (25%, 57.4 mL) was added drop-wise over 15 minutes to 20 minutes. Ethanol (25 mL) was added to the mixture. The mixture was stirred at 15° C. to 20° C. for 15 minutes to 20 minutes. (S)-Epichlorohydrin (50 g, 0.540 moles) and ethanol (50 mL) were added. The reaction mixture was heated to 40° C. and stirred for 1 hour at 15° C. to 40° C. The reaction mixture was again stirred at 35° C. to 40° C. for 6 hours, cooled to 25° C. to 30° C., and further stirred for 12 hours. The solution was concentrated to dryness under vacuum at 50° C. to 55° C. Ethanol (50 mL) was added to the oil obtained, and the mixture was concentrated under vacuum at 50° C. to 55° C. Toluene (125 mL) was added to the oil obtained, and the mixture was heated to 35° C. to 40° C. Aqueous hydrochloric acid (6.8 N, 129.5 mL) was added to the solution at 35° C. to 40° C. and stirred for 2 hours. The reaction mass was cooled to 25° C. to 30° C., and the aqueous layer was separated. The organic layer was extracted with water (50 mL). The combined aqueous layers were concentrated under vacuum at 70° C. to 75° C. to get a semi-solid material. The semi-solid material was charged with ethanol (25 mL) and heated to 60° C. to 65° C. to get a clear solution. The solution was first cooled to 25° C. to 30° C. and then to −20° C. The slurry obtained was stirred for 1 hour at −20° C. The slurry was filtered and suck dried. The wet solid was dried at 45° C. to 50° C. under vacuum.

Yield=31.5 g (50%)

### Example 2

### Preparation of 5-chloro-N-[(2S)-3-chloro-2-hydroxypropyl]thiophene-2-carboxamide (Formula III)

Sodium bicarbonate (11.1 g, 0.132 moles) was added to a solution of (2S)-1-amino-3-chloropropan-2-ol hydrochloride (of Example 1; 15 g, 0.102 moles) in tetrahydrofuran (45 mL) and deionized water (90 mL) at ambient temperature. The mixture was stirred at 25° C. to 30° C. for 10 minutes to 15 minutes. The mixture was cooled to 15° C. and a solution of 5-chlorothiophene-2-carbonylchloride (24 g, 0.132 moles) in toluene (22.5 mL) was added at 10° C. to 15° C. over 30 minutes to 35 minutes. The mixture was stirred at 10° C. to 15° C. for 2 hours and the reaction mass was heated to 25° C. to 30° C. The organic layer was separated and the aqueous layer was extracted with toluene (45 mL). The combined organic layers were concentrated in vacuum at 45° C. to 50° C. to get a brown colored solid. The solid was suspended in toluene (75 mL). The suspension was heated to 45° C. to 50° C. and stirred at 45° C. to 50° C. for 15 minutes. The mixture was cooled to 25° C. to 30° C. and stirred at 25° C. to 30° C. for 2 hours. The slurry obtained was filtered, washed with toluene (10 mL), and the wet solid obtained was dried at 50° C. to 55° C. under vacuum.

Yield=19.0 g (75%)

Melting Point=107° C. to 109° C.

MS (m/z)=254

### Example 3

### Preparation of (2S)-1-chloro-3-{[(5-chlorothiophen-2-yl)carbonyl]amino}propan-2-yl[4-(3-oxomorpholin-4-yl)phenyl]carbamate (Formula II)

Pyridine (0.9315 g, 0.01179 moles) was added to a solution of 5-chloro-N-[(2S)-3-chloro-2-hydroxypropyl]thiophene-2-carboxamide (Formula III; 1 g, 0.00393 moles) in dichloromethane (5 mL) at 25° C. to 30° C. and then cooled to 10° C. The mixture was stirred for 15 minutes at 10° C. to 15° C. A solution of triphosgene (0.290 g, 0.00097 moles) in dichloromethane (5 mL) was added slowly to the mixture at 10° C. to 15° C. and the mixture was stirred for 1 hour at 10° C. to 15° C. Pyridine (0.311 g, 0.00393 moles), 4-(4-aminophenyl)morpholin-3-one (Formula IV; 0.568 g, 0.002925 moles) and dimethylamino pyridine (0.10 g, 0.00818 moles) were added to the reaction mass at 10° C. to 15° C. The reaction mass was allowed to reach 20° C. to 25° C. and was stirred for 2 hours at 20° C. to 25° C. The resulting mass was quenched with deionized water (5 mL) at 20° C. to 25° C. The organic layer was separated and washed with deionized water (3×5 mL). The organic layer was concentrated under vacuum at 30° C. to 35° C. to get a solid material. The solid material was crystallized in ethyl acetate (2 mL) and hexane (5 mL). The slurry obtained was filtered and suck dried. The wet solid was dried under vacuum at 50° C. to 55° C.

Yield=1 g (50%)

Melting Point=65° C. to 70° C.

MS (m/z)=472

### Example 4

### Preparation of Rivaroxaban (Formula I)

Potassium carbonate (0.135 g, 0.000952 moles) was added to a solution of (2S)-1-chloro-3-{[(5-chlorothiophen-2-yl)carbonyl]amino}propan-2-yl[4-(3-oxomorpholin-4-yl)phenyl]carbamate (Formula II; 0.3 g, 0.000635 moles) in acetone (6 mL) and deionized water (3 mL) at 25° C. to 30° C. The mixture was stirred for 12 hours at 25° C. to 30° C. The reaction mixture was extracted with dichloromethane (10 mL). The organic layer was separated and concentrated under vacuum to get an oily product. The oily product was crystallized in ethyl acetate (3 mL) and hexanes (5 mL) at 25° C. to 30° C. The slurry obtained was filtered and suck dried. The wet solid was dried under vacuum at 40° C. to 45° C.

Yield=0.15 g (56%)

We claim:

1. A process for the preparation of a compound of Formula II, 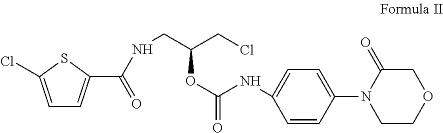 [SMILES: O=C(Nc1ccc(N2CCOCC2=O)cc1)O[C@H](CCl)CNC(=O)c1ccc(Cl)s1]
wherein the process comprises treating a compound of Formula III 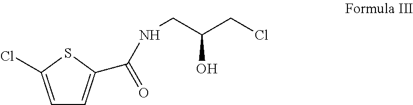 [SMILES: O=C(NC[C@H](O)CCl)c1ccc(Cl)s1]
with a compound of Formula IV 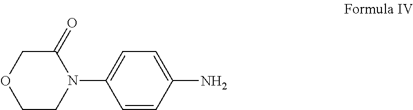 [SMILES: Nc1ccc(N2CCOCC2=O)cc1]
in the presence of phosgene or a phosgene equivalent.

2. A process for the preparation of rivaroxaban of Formula I, 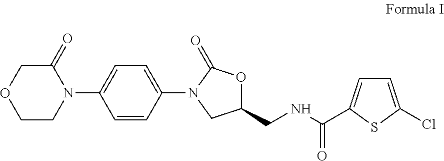 [SMILES: O=C(NC[C@H]1CN(c2ccc(N3CCOCC3=O)cc2)C(=O)O1)c1ccc(Cl)s1]
wherein the process comprises cyclization of a compound of Formula II 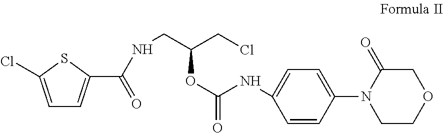 [SMILES: O=C(Nc1ccc(N2CCOCC2=O)cc1)O[C@H](CCl)CNC(=O)c1ccc(Cl)s1]
to obtain rivaroxaban of Formula I.

3. A process for the preparation of rivaroxaban of Formula I, 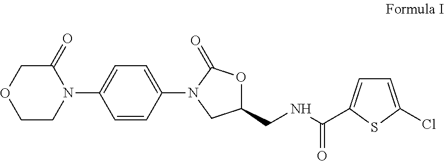 [SMILES: O=C(NC[C@H]1CN(c2ccc(N3CCOCC3=O)cc2)C(=O)O1)c1ccc(Cl)s1]
wherein the process comprises:
a) treating a compound of Formula III 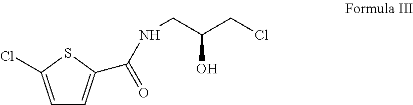 [SMILES: O=C(NC[C@H](O)CCl)c1ccc(Cl)s1]
with a compound of Formula IV 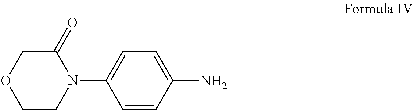 [SMILES: Nc1ccc(N2CCOCC2=O)cc1]
in the presence of phosgene or a phosgene equivalent to obtain a compound of Formula II; and 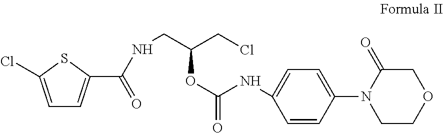 [SMILES: O=C(Nc1ccc(N2CCOCC2=O)cc1)O[C@H](CCl)CNC(=O)c1ccc(Cl)s1]
b) cyclization of the compound of Formula II 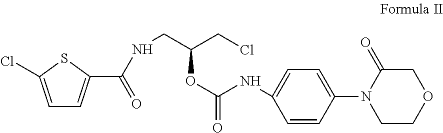 [SMILES: O=C(Nc1ccc(N2CCOCC2=O)cc1)O[C@H](CCl)CNC(=O)c1ccc(Cl)s1]
to obtain rivaroxaban of Formula I.

4. The process according to claim 1 or 3, wherein the compound of Formula III is treated with the compound of Formula IV in a solvent in the presence of a base.

5. The process according to claim 4, wherein the base is selected from pyridine, dimethylaminopyridine, triethylamine, sodium carbonate, potassium carbonate, or mixtures thereof.

6. The process according to claim 1 or 3, wherein a solution of phosgene or a phosgene equivalent in a solvent is added to a mixture containing the compound of Formula III and a base in a solvent prior to the treatment of the compound of Formula III with the compound of Formula IV.

7. The process according to claim 6, wherein the base is selected from pyridine, triethylamine, sodium carbonate, potassium carbonate, or a mixture thereof.

8. The process according to claim 4 or 6, wherein the solvent is selected from dichloromethane, dichloroethane, or a mixture thereof.

9. The process according to claim 1, 3 or 6, wherein the phosgene equivalent is selected from diphosgene, triphosgene, carbonyldiimidazole or disuccinimidyl carbonate.

10. The process according to claim 2 or 3, wherein the compound of Formula II is cyclized in a solvent in the presence of a base.

11. The process according to claim 10, wherein the solvent is selected from acetone, acetonitrile, methanol, ethanol, isopropanol, dioxane, tetrahydofuran, water, or a mixture thereof.

12. The process according to claim 10, wherein the base is selected from potassium carbonate, potassium bicarbonate, potassium hydroxide, sodium hydroxide, sodium carbonate, sodium bicarbonate, sodium hydride, or a mixture thereof.

13. A compound of Formula II.

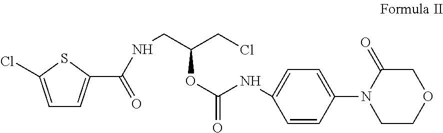 [SMILES: O=C(Nc1ccc(N2CCOCC2=O)cc1)O[C@H](CCl)CNC(=O)c1ccc(Cl)s1]

14. Use of a compound of Formula II 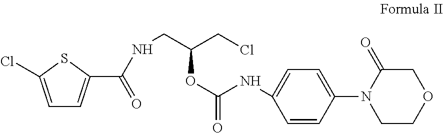 [SMILES: O=C(Nc1ccc(N2CCOCC2=O)cc1)O[C@H](CCl)CNC(=O)c1ccc(Cl)s1]
for the preparation of rivaroxaban.
